(19) United States
(12) Patent Application Publication
(10) Pub. No.: US 2007/0009589 A1
(43) Pub. Date: Jan. 11, 2007

(54) EXTENDED RELEASE COMPOSITIONS

(76) Inventors: Kandarapu Raghupathi, Hyderabad 500 050, A.P. (IN); Mohammad Bala Pasha, Hyderabad 500 050, A.P. (IN); Vijay Dinanathji Nasare, Nagpur Katol 441 302, Maharashtra (IN); Indu Bhushan, Hyderabad 500 072, A.P. (IN); Mailatur Sivaraman Mohan, Hyderabad 500 072, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/428,936

(22) Filed: Jul. 6, 2006

Related U.S. Application Data
(60) Provisional application No. 60/718,785, filed on Sep. 20, 2005.

(30) Foreign Application Priority Data
Jul. 7, 2005 (IN) 889/CHE/2005

(51) Int. Cl. A61K 9/48; A61K 9/20

(52) U.S. Cl. 424/451; 424/464

(57) ABSTRACT

Pharmaceutical compositions of metoprolol or a salt have water-insoluble inorganic cores such as dibasic calcium phosphate having the drug deposited thereon, optionally with one or more hydrophilic or hydrophobic polymers or mixtures thereof, and an outer coating of a polymer blend utilizing groups of polymers having opposing wettability characteristics.

The present invention relates to extended release pharmaceutical compositions of drug compounds.

Metoprolol, a cardioselective adrenoreceptor-blocking agent, is a highly lipophilic drug having Log P 2.48. Because of low water solubility, its pharmaceutically acceptable salts like metoprolol tartrate and metoprolol succinate are preferred for oral formulations.

Metoprolol succinate is chemically (±)-1-(isopropylamino)-3-[p-(2-methoxyethyl) phenoxy]-2-propanol succinate (2:1). It is freely soluble in water and useful in the treatment of hypertension, angina pectoris and heart failure. It is commercially available in extended release tablets under the brand name TOPROL XL® and is manufactured by AstraZeneca. The structural formula for metoprolol succinate is Formula I.

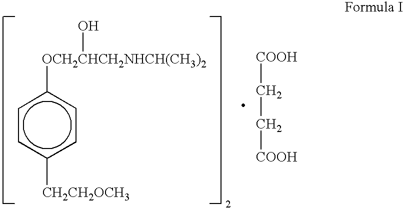

Extended release drug delivery systems are useful in delivering active pharmaceutical ingredients that have one or more of a narrow therapeutic range, short biological half-life and high toxicities. These systems allow the dosage delivery by reducing the number of administrations and provide the desired therapeutic effect throughout the day.

U.S. Pat. Nos. 4,927,640 and 4,957,745 and U.S. Patent Application Publication Nos. 2005/0008701 and 2003/0185887 describe controlled release preparations of metoprolol salts and methods for the production thereof.

U.S. Patent Application Publication No. 2005/0181049 discloses compositions and methods for enhancing bioavailability of drugs with low water solubility (Biopharmaceutic Classification System Class 2), wherein such solubility-problem drug, along with hydrophilic polymer, is applied to a porous substrate including magnesium aluminometasilicate, anhydrous dibasic calcium phosphate, microcrystalline cellulose, and the like. The assembly of drug-polymer complex and porous carriers dissociates in vivo to set free the drug-polymer complex, which further dissociates to release drug in an aqueous environment.

Most of the extended delivery systems for metoprolol succinate are based on use of water-soluble or water swellable organic seed cores. Water solubility or swellability of the core may lead to disruption of the structural integrity of the coating. Such a disruption of coating with a water-soluble core might cause unpredictable drug release pattern and hence dose dumping. In addition to stability problems, organic seed cores are also more prone to react with the drug or polymer. Hence there is a need to formulate extended delivery systems for metoprolol succinate using inorganic seed cores.

### SUMMARY OF THE INVENTION

The present invention relates to extended release pharmaceutical compositions of metoprolol or its pharmaceutically acceptable salts, solvates, polymorphs, enantiomers, single isomer, or mixtures thereof.

More particularly, an embodiment of this invention relates to a pharmaceutical composition having:

a. water-insoluble inorganic seed core comprising dibasic calcium phosphate;

b. metoprolol or its pharmaceutically acceptable salt, optionally with one or more hydrophilic or hydrophobic polymer or mixtures thereof, deposited or layered or applied onto the said seed core; and

c. optionally an outer coat of polymer blend utilizing groups of polymers having opposing wettability characteristics; that releases metoprolol or its pharmaceutically acceptable salt substance in a extended manner over a period of time.

In an aspect, the invention includes a pharmaceutical composition comprising an inert water-insoluble particle having a first coating comprising a drug substance, and optionally a second coating disposed over the first coating and containing a mixture of hydrophilic and hydrophobic polymers.

In another aspect, the invention includes a pharmaceutical composition comprising an inert water-insoluble particle having a first coating comprising metoprolol or a salt thereof, and optionally a hydrophilic polymer, a hydrophobic polymer, or a mixture thereof, and a second coating disposed over the first coating and containing a mixture of hydrophilic and hydrophobic polymers.

In a further aspect, the invention includes a pharmaceutical composition comprising multiple particles comprising a dibasic calcium phosphate having a first coating comprising metoprolol or a salt thereof and a hydrophilic polymer, a hydrophobic polymer, or a mixture thereof, and a second coating disposed over the first coating and containing a mixture of hydrophilic and hydrophobic polymers.

### DETAILED DESCRIPTION OF THE INVENTION

The present invention relates to extended release pharmaceutical compositions of metoprolol or its pharmaceutically acceptable salts, solvates, polymorphs, enantiomers, single isomer, or mixtures thereof.

More particularly this invention relates to a pharmaceutical composition having:

a. water-insoluble inorganic seed core comprising dibasic calcium phosphate;

b. metoprolol or its pharmaceutically acceptable salt, optionally with one or more hydrophilic or hydrophobic polymer or mixtures thereof, deposited or layered or applied onto the said seed core; and

c. optionally an outer coat of polymer blend utilizing groups of polymers having opposing wettability characteristics;

that releases metoprolol or its pharmaceutically acceptable salt substance in a extended manner over a period of time.

The present invention solves the problems associated with the use of a water-soluble and/or water swellable cores for extended release pellets or beads by making use of a water-insoluble inorganic seed core which is neither water-soluble nor water swellable. The water-soluble seed cores cause the controlled release pellets to burst and dump the drug, which is not the case with the present invention.

Achieving an extended release from a seed core formulation depends on the integrity of seed core and the type of release controlling coat on the seed core. In case of water-soluble seed core, the core dissolves as the dissolution process progresses leading to the disruption of the structural integrity of the coating. Such a disruption of coating with a water-soluble core might cause unpredictable drug release pattern and hence dose dumping. On the contrary, in case of the water-insoluble core, the coating integrity is maintained throughout the dissolution period due to structurally undisturbed seed core resulting in a predictable dissolution profile of the product.

The compositions comprise a large number of small inorganic insoluble particle cores that are covered by pharmaceutical active substance. The size of cores range from 50-5000 μm, or in the range of 100-500 μm, or ranges from 150-300 μm. The cores are inert, pharmaceutically compatible, inorganic, and water insoluble in nature. Examples of various substances that can be used as inert inorganic cores are calcium carbonate, dibasic calcium phosphate anhydrous, dibasic calcium phosphate monohydrate, tribasic calcium phosphate, magnesium carbonate, magnesium oxide and the like. This list is not exhaustive, as other substances are also suitable for forming the cores.

The inert water-insoluble inorganic seed core being an inorganic excipient is non-reactive and compatible with the active and the inactive excipients, does not need an additional coating as is the case with water-soluble cores and is more economical and undergoes complete coating with less dust generation in fluidized-bed coater.

Dibasic calcium phosphate is a widely used pharmaceutical excipient, which is available in two forms, hydrous and anhydrous. Depending on the moisture sensitivity of the drug, any one of the forms can be chosen. In one of the embodiments, dibasic calcium phosphate anhydrous has been found to be useful as inert water-insoluble inorganic seed core. Dibasic calcium phosphate anhydrous being an inorganic excipient is compatible with the active and the inactive excipients. Moreover, being water-insoluble, it does not need an additional coating as is the case with water-soluble cores and thus is more economical. Also its density is less than that of glass beads and more than that of plastic-resin particles, and thus undergoes coating completely in the fluidized-bed coater. Dibasic calcium phosphate anhydrous also generates low dust during coating in the fluidized-bed coater. Owing to the above-mentioned criteria, dibasic calcium phosphate anhydrous has been found to be particularly useful as an inert water-insoluble seed core for the present invention.

The present invention in one embodiment provides metoprolol succinate on inert water-insoluble seed cores, coated with a hydrophilic-hydrophobic swellable coating material of a defined coating built up. Although metoprolol succinate is discussed with particularity herein, other salts and many other drug substances can be used in the invention, and the invention is not limited to only metoprolol succinate compositions.

The said system comprises a hydrophilic-hydrophobic swellable coating composition, wherein the composition controls the release of metoprolol succinate.

The hydrophilic-hydrophobic swellable coating composition comprises various hydrophilic polymers having a high degree of swelling in aqueous fluids. Such hydrophilic polymers of various grades are exemplified but are not limited to, celluloses such as carboxymethyl cellulose sodium, hydroxyethyl cellulose, hydroxypropyl methylcellulose (HPMC); homopolymers or copolymers of N-vinylpyrrolidone; vinyl and acrylic polymers; polyacrylic acid and the like; hydrophobic polymers such as celluloses like ethyl cellulose, low substituted hydroxypropyl cellulose (L-HPC), cellulose acetate, cellulose propionate (lower, medium or higher molecular weight), cellulose acetate propionate, cellulose acetate butyrate, cellulose acetate phthalate; polyalkyl methacrylates; polyalkyl acrylates; polyvinyl acetate (PVA); chitosan; stearic acid, gum arabic, crosslinked vinylpyrrolidone polymers; hydrogenated castor oil; and the like. Other classes of rate controlling substances or their mixtures in various ratios as required are also within the purview of this invention without limitation.

Of course, any other polymer, which demonstrates such characteristics and is useful for the coating composition to modulate the release of the metoprolol, is also acceptable in the working of this invention.

In one of the embodiments, polymers simultaneously possessing swelling and gelling properties such as hydroxypropyl methylcellulose have been found particularly useful for the coating composition in combination with hydrophobic polymers such as ethyl cellulose to modulate the release of the metoprolol in a predictable extended manner for a prolonged or sustained period of time.

According to the present invention, the ratio of the hydrophilic to hydrophobic material for the coating composition ranges from 1:9 to 9:1, or from 1:5 to 5:1 and or from 1:3 to 3:1.

The water-insoluble inorganic seed core of active substance(s) coated with rate controlling polymers can be formulated as tablets, beads filled into hard gelatin capsules, sachets and the like to obtain the desired in vivo release profiles after administration.

In context of the present invention, during the preparation of the pharmaceutical compositions into finished dosage form, one or more pharmaceutically acceptable excipients may optionally be used which include but are not limited to: diluents such as microcrystalline cellulose (MCC), silicified MCC (e.g. Prosolv™ HD 90), microfine cellulose, lactose, starch, pregelatinized starch, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like; binders such as acacia, guar gum, alginic acid, dextrin, maltodextrin, methylcellulose, ethyl cellulose, hydroxyethyl cellulose, hydroxypropyl cellulose (e.g. KLUCEL®), hydroxypropyl methylcellulose (e.g. METHOCEL®), carboxymethyl cellulose sodium, povidone (various grades of KOLLIDON®, PLASDONE®), starch and the like; disintegrants such as carboxymethyl cellulose sodium (e.g. Ac-Di-Sol®, Primellose®), crospovidone (e.g. Kollidon®, Polyplasdone®), povidone K-30, polacrilin potassium, starch, pregelatinized starch, sodium starch glycolate (e.g. Explotab®) and the like; surfactants including anionic surfactants such as chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, sodium lauryl sulfate (SLS) and sodium dodecyl sulfate (SDS); cationic surfactants such as cetylpyridinium chloride monohydrate and hexadecyltrimethylammonium bromide; nonionic surfactants such as N-decanoyl-N-methylglucamine, octyl a-D-glucopyranoside, n-Dodecyl b-D-maltoside (DDM), polyoxyethylene sorbitan esters like polysorbates and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like; solvents that may be used in granulation or layering or coating are such as aqueous like water or alcoholic like ethanol, isopropanolol or hydro-alcoholic like a mixture of water with alcohol in any ratio or organic like acetone, methylene chloride, dichloromethane and the like.

Pharmaceutical compositions of the present invention may further include any one or more of pharmaceutically acceptable glidants, lubricants, opacifiers, colorants and other commonly used excipients.

The present invention will provide a unit dose of metoprolol of about 10 to about 250 milligrams per dosage form.

In the context of the present invention, release of active may be achieved by means of formulating the active substance using matrix or reservoir or combination of matrix-reservoir principles and the active may be further presented as monolithic or as multi particulate compositions.

Pharmaceutical fixed dose compositions based on a matrix principle may be prepared by direct blending, dry granulation or wet granulation of active substance with one or more rate modifying substances and they are filled into capsules or compressed as tablets or layered on to inert beads and further such beads are filled into capsules or compressed as tablets.

Pharmaceutical fixed dose compositions based on a reservoir principle may be prepared by coating the powders or granules or pellets or tablets or cores with one or more rate modifying substances and they may be filled into capsules.

Pharmaceutical fixed dose compositions may also be prepared using matrix-reservoir principles by first preparing the matrix portion as mentioned in the previous paragraphs and subsequently coating the matrix composition with one or more rate modifying substances.

Rate and extent of release of active from the composition depends on the type and amount of rate modifying substance(s) used, the type of composition used and the processes used to prepare the compositions.

The pharmaceutical fixed dose compositions can further be optionally film coated or enteric coated or seal coated or coated with substances to modify the release of the active. The coating can be done by techniques known to one skilled in the art such as powder coating, spray coating, dip coating, fluidized bed coating and the like.

The pharmaceutical compositions of the present invention can also be manufactured as described below. The granules or cores can be prepared by sifting the active and excipients through the desired mesh size sieve and then mixed using a rapid mixer granulator, planetary mixer, mass mixer, ribbon mixer, fluid bed processor or any other suitable device. The blend can be granulated by dry or wet granulation. In wet granulation, the granulate can be dried using a tray drier, fluid bed drier, rotary cone vacuum drier and the like. The dried granulate particles are sieved and then mixed with lubricants and disintegrants and compressed into tablets or filled into capsules.

Further, the manufacture of granules may be done by direct compression with the use of directly compressible excipients using a suitable device, such as a multi-station rotary machine, to form compressed slugs or by roller compaction to form slugs, which are passed through a multimill, fluid energy mill, ball mill, colloid mill, roller mill, hammer mill and the like, equipped with a suitable screen. The milled slugs are then lubricated and compressed into tablets or pellets and are coated with a rate controlling substance. Coated pellets are further filled into capsules or compressed as tablets.

Metoprolol succinate is applied to the water-insoluble inorganic seed core by any conventional techniques such as but not limited to pan coating, roto-granulation, fluidized bed coating and the like.

The present invention solves the problems associated with the use of water-soluble and/or water swellable cores for metoprolol extended release formulations. The extended release compositions are prepared by coating a drug layer or layers onto water-insoluble inorganic seed core and then applying over the drug layer a polymeric coating that controls the release of the drug. The said extended release compositions are then formed into an extended release tablet or capsule for oral administration.

The pharmaceutical compositions as disclosed in context of the present invention are useful in the treatment of hypertension.

The following examples will further exemplify certain specific aspects and embodiments of the invention, and are not to be construed as limiting the scope of the invention.

### EXAMPLE 1

### Compositions for Metoprolol Extended Release (“ER”) Tablets

| | 200 mg/ | 100 mg/ | 50 mg/ | 25 mg/ |
|---|---|---|---|---|
| Ingredient | unit | unit | unit | unit |
| Dibasic calcium phosphate | 56.2 | 28.1 | 14.1 | 7.0 |
| anhydrous (60/80 mesh) | | | | |

Drug loading

| | | | | |
|---|---|---|---|---|
| Metoprolol succinate* | 190 | 95 | 47.5 | 23.7 |
| Hydroxypropyl methylcellulose, | 3.7 | 1.9 | 0.9 | 0.5 |
| 5 cPS | | | | |

Release retarding coat

| | | | | |
|---|---|---|---|---|
| Ethyl cellulose, 10 cPS | 135.7 | 67.9 | 33.9 | 16.9 |
| Hydroxypropyl methylcellulose, | 40.7 | 20.3 | 10.2 | 5.1 |
| 5 cPS | | | | |
| Acetyltributyl citrate | 32.5 | 16.3 | 8.1 | 4.1 |

Granulation

| | | | | |
|---|---|---|---|---|
| Prosolv HD 90 (Silicified MCC)# | 394.5 | 197.3 | 140.7 | 70.3 |
| Hydroxypropyl cellulose LF | 39.4 | 19.7 | 12.8 | 6.4 |

Blending and lubrication

| | | | | |
|---|---|---|---|---|
| Colloidal silicon dioxide | 28.2 | 14.1 | 7 | 3.5 |
| (Aerosil ™ 200) | | | | |
| Sodium stearyl fumarate | 4.7 | 2.3 | 1.2 | 0.6 |
| Croscarmellose sodium | 14.1 | 7 | 3.5 | 1.8 |

Coating

| | | | | |
|---|---|---|---|---|
| Hydroxypropyl methylcellulose, | 16.5 | 8.3 | 5.1 | 2.6 |
| 5 cPS | | | | |
| Polyethylene glycol 6000 | 24.8 | 12.4 | 6.2 | 3.1 |
| Talc | 2.06 | 1 | 0.5 | 0.2 |
| Titanium dioxide | 16.6 | 8.3 | 4.1 | 2.1 |

*Amounts expressed as their metoprolol tartrate equivalents
#Silicified microcrystalline cellulose (or co-processed MCC with silicon dioxide), JRS Pharma GmbH Co. KG, Rosenberg, Germany

Manufacturing Process
1. Hydroxypropylmethyl cellulose was dispersed in water while stirring with the help of a mechanical stirrer. Accurately weighed amount of metoprolol succinate was added to this polymeric dispersion and stirred to form a dispersion.
2. The drug-polymeric dispersion so formed was coated on dibasic calcium phosphate particles (60/80 mesh) till desired dose of the drug is built up using a fluidized bed coater (FBC).
3. Using acetyltributyl citrate as a plasticizer, uniform dispersion of ethyl cellulose and hydroxypropyl methylcellulose was prepared with the help of the mechanical stirrer in a mixture of isopropyl alcohol and methylene chloride.
4. On the drug-loaded pellets, release-modifying polymeric dispersion of step 3 was applied by coating in a fluidized bed coater (FBC). Coated pellets were dried in the FBC at 60±5° C. for 2 hours.
5. Granules of hydroxypropyl cellulose as an aqueous binder and Prosolv HD 90 were prepared using top-spray technique of fluid bed coater (FBC). Drying was done until loss on drying ranged between 0.5% w/w to 1.5% w/w.
6. The coated pellets of step 4 were blended with Prosolv HD 90 granules (of step 5), Aerosil 200, sodium stearyl fumarate and croscarmellose sodium in a double cone blender for 5 minutes.
7. The blended pellets were compressed using 19×9.5 mm modified capsule shaped punches with corresponding dies.
8. Finally, the compressed tablets were film coated in a Neocoater with hydroxypropyl methylcellulose containing titanium dioxide as an opacifier, PEG 6000 as a plasticizer and talc as an anti-adherent, dispersed in a mixture of isopropyl alcohol and methylene chloride.

### EXAMPLE 2

Dissolution profile of compositions for metoprolol succinate extended release tablets of Example 1 (200 mg).

Dissolution media: pH 6.8 phosphate buffer

Apparatus: USP type 2

Stirring speed: 50 rpm.

Volume: 500 mL

Temperature: 37.5±0.5° C.

| | Metoprolol ER Tablets 200 mg |
|---|---|
| | of Example 1 |
| Time (hr) | % Drug Dissolved |
| 1 | 10 |
| 4 | 31 |
| 8 | 49 |
| 12 | 62 |
| 20 | 85 |

### EXAMPLE 3

### Composition of Metoprolol Extended Release Tablets 200 mg

| | |
|---|---|
| | Grams per |
| | batch of 1000 |
| Ingredient | tablets |

Seal-coating

| | |
|---|---|
| Dicalcium phosphate anhydrous | 33 |
| Ethyl cellulose 10 cPS* | 4 |
| Acetyltributyl citrate | 1 |
| Isopropyl alcohol | 47.5 |
| Methylene chloride | 47.5 |

Drug-loading

| | |
|---|---|
| Metoprolol succinate | 190 |
| Hydroxypropyl methylcellulose, 5 cPS | 22 |
| Water | 600 |
| Weight of drug-loaded pellets | 250 |

ER coating

| | |
|---|---|
| Ethyl cellulose, 10 cPS | 120 |
| Hydroxypropyl methylcellulose, 5 cPS | 26 |
| Acetyltributyl citrate | 29 |
| Isopropyl alcohol | 2000 |
| Methylene chloride | 1000 |
| Weight of ER coated pellets (A) | 425 |

Granulation

| | |
|---|---|
| Prosolv HD 90 (Silicified MCC) | 416.3 |
| Hydroxypropyl cellulose (Klucel LF) | 40.5 |
| Water | 640 |

Lubrication

| | |
|---|---|
| Hydroxypropyl cellulose (Klucel LF) | 30 |
| Croscarmellose sodium | 23.5 |
| Sodium stearyl fumarate | 4.7 |
| Placebo blend weight (B) | 515 |
| Hydroxypropyl methylcellulose, 5 cPS | 16.6 |
| Polyethylene glycol 6000 | 24.8 |
| Talc | 2.1 |
| Titanium dioxide | 16.6 |
| Isopropyl alcohol | 570 |
| Methylene chloride | 570 |
| Film coating weight (C) | 60 |
| Theoretical weight of tablet (A + B + C) | 1000 |

Manufacturing Process:
1. Ethyl cellulose and acetyltributyl citrate were dispersed in a mixture of isopropyl alcohol and methylene chloride.
2. The dispersion of step 1 was coated onto dibasic calcium phosphate using a fluidized bed coater (FBC) till desired weight built up was obtained.
3. Further process for drug loading, ER coating, granulation, lubrication, compression and top coating is similar to that of Example 1.
   Dissolution Profile:

Media: pH 6.8 phosphate buffer

Apparatus: USP type 2

Stirring speed: 50 rpm

Volume: 500 mL

Temperature: 37.5±0.5° C.

| | Metoprolol ER Tablets 200 mg of |
|---|---|
| | Example 3 |
| Time (hr) | % Drug Dissolved |
| 0 | 0 |
| 1 | 9 |
| 4 | 28 |
| 8 | 44 |
| 20 | 91 |

### EXAMPLE 4

### Composition of Metoprolol Extended Release Tablets 200 mg

| | |
|---|---|
| | Grams per |
| | batch of 1000 |
| Ingredient | tablets |

Drug loading

| | |
|---|---|
| Dicalcium phosphate anhydrous | 49.5 |
| Ethyl cellulose 10 cPS* | 3 |
| Metoprolol succinate | 190 |
| Hydroxypropyl methylcellulose, 5 cPS | 7.5 |
| Isopropyl alcohol | 45 |
| Methylene chloride | 45 |
| Water | 500 |
| Weight of drug-loaded pellets | 250 |

ER coating

| | |
|---|---|
| Ethyl cellulose, 10 cPS | 142.9 |
| Hydroxypropyl methylcellulose, 5 cPS | 42.9 |
| Acetyltributyl citrate | 34.3 |
| Isopropyl alcohol | 1200 |
| Methylene chloride | 1200 |
| Weight of ER coated pellets (A) | 470 |

Placebo granules

| | |
|---|---|
| Prosolv HD 90 (Silicified MCC) | 413.6 |
| Hydroxypropyl cellulose LF | 28.2 |
| Water | 560 |
| Croscarmellose sodium | 23.5 |
| Sodium stearyl fumarate | 4.7 |
| Compression placebo weight (B) | 470 |
| Compressed tablet weight (mg) | 940 |

Film coating

| | |
|---|---|
| Hydroxypropyl methylcellulose, 5 cPS | 16.6 |
| Polyethylene glycol 6000 | 24.8 |
| Talc | 2.1 |
| Titanium dioxide | 16.6 |
| Isopropyl alcohol | 580 |
| Methylene chloride | 580 |
| Film coating weight (C) | 60 |
| Theoretical weight of tablet (A + B + C) | 1000 |

Manufacturing process: Similar to that described in Example 3.
Dissolution Profile:

Media: pH 6.8 phosphate buffer

Apparatus: USP type 2

Stirring speed: 50 rpm

Volume: 500 mL

Temperature: 37.5±0.5° C.

| | Metoprolol Succinate ER |
|---|---|
| | Tablets 200 mg Example 4 |
| Time (hr) | % Drug Dissolved |
| 0 | 0 |
| 1 | 8 |
| 4 | 30 |
| 8 | 53 |
| 12 | 71 |
| 20 | 92 |

### EXAMPLE 5

### Composition of Metoprolol Extended Release Tablets 200 mg Comprising Surelease in ER Coating

| | |
|---|---|
| | Grams per |
| | batch of 1000 |
| Ingredient | tablets |

Drug loading

| | |
|---|---|
| Dicalcium phosphate anhydrous | 18 |
| Ethyl cellulose 10 cPS | 2 |
| Metoprolol succinate | 190 |
| Isopropyl alcohol | 50 |
| Methylene chloride | 50 |
| Water | 600 |
| Weight of drug-loaded pellets | 210 |

ER coating

| | |
|---|---|
| Surelease* E719010 | 81 |
| Water | 1500 |
| Weight of ER coated pellets (A) | 291 |

Placebo granules

| | |
|---|---|
| Prosolv HD 90 (Silicified MCC) | 465 |
| Hydroxypropyl cellulose LF | 20 |
| Water | 500 |
| Croscarmellose sodium | 20 |
| Sodium stearyl fumarate | 4 |
| Compression placebo weight (B) | 509 |
| Compressed tablet weight (mg) | 800 |

Film coating

| | |
|---|---|
| Hydroxypropyl methylcellulose, 5 cPS | 16.6 |
| Polyethylene glycol 6000 | 24.8 |
| Talc | 2.1 |
| Titanium dioxide | 16.6 |
| Isopropyl alcohol | 500 |
| Methylene ahloride | 500 |
| Film coating weight (C) | 60 |
| Theoretical weight of tablet (A + B + C) | 860 |

Surelease ™ E719010 is a proprietary sustained release ethyl cellulose dispersion coating composition from Colorcon, West Point, Pennsylvania.

Manufacturing Process:
1. Drug loading: Metoprolol and polymers were dispersed in the solvent mixture of isopropyl alcohol and methylene chloride. This dispersion was coated onto dibasic calcium phosphate using FBC till desired weight built up was obtained.
2. ER coating: The polymers were dispersed in the mixture of isopropyl alcohol and methylene chloride prepared with the help of the mechanical stirrer. This dispersion was coated onto drug loaded dibasic calcium phosphate cores using FBC.
3. Granulation: Placebo granules of Prosolv and hydroxypropyl cellulose were prepared by standard aqueous wet granulation method using top-spray technique of fluid bed coater (FBC). Drying was done until loss on drying ranged between 0.5% w/w to 1.5% w/w.
4. The placebo granules of step 3 and ER coated cores of step 2 were blended with croscarmellose sodium and sodium stearyl fumarate in a double cone blender for 5 minutes.

5. The blended pellets were compressed using 19×9.5 mm modified capsule shaped punches with corresponding dies.

6. Finally, the compressed tablets were film coated in a Neocoater with hydroxypropyl methylcellulose containing titanium dioxide as an opacifier, PEG 6000 as a plasticizer and talc as an anti-adherent.

### EXAMPLE 6

### Composition of Metoprolol Extended Release Tablets 200 mg with ER Coating Comprising Hydrophilic and Hydrophobic Polymer Mixture

| | |
|---|---|
| | Grams per |
| | batch of 1000 |
| Ingredient | tablets |

Drug loading

| | |
|---|---|
| Dicalcium phosphate anhydrous | 18 |
| Ethyl cellulose 10 cPS | 2 |
| Metoprolol succinate | 190 |
| Isopropyl alcohol | 65 |
| Methylene chloride | 65 |
| Water | 580 |
| Weight of drug-loaded pellets | 210 |

ER coating

| | |
|---|---|
| Ethyl cellulose, 10 cPS | 60.7 |
| Hydroxypropyl methylcellulose, 5 cPS | 15.2 |
| Acetyltributyl citrate | 12.1 |
| Isopropyl alcohol | 1100 |
| Methylene chloride | 1100 |
| Water | 285 |
| Weight of ER coated pellets (A) | 298 |

Placebo granules

| | |
|---|---|
| Prosolv HD 90 (Silicified MCC) | 582 |
| Hydroxypropyl cellulose LF | 31.8 |
| Water | 550 |
| Croscarmellose sodium | 23.5 |
| Sodium stearyl fumarate | 4.7 |
| Compression placebo weight (B) | 642 |
| Compressed tablet weight (mg) | 940 |

Film coating

| | |
|---|---|
| Hydroxypropyl methylcellulose, 5 cPS | 16.6 |
| Polyethylene glycol 6000 | 24.8 |
| Talc | 2.1 |
| Titanium dioxide | 16.6 |
| Isopropyl alcohol | 500 |
| Methylene chloride | 500 |
| Film coating weight (C) | 60 |
| Theoretical weight of tablet (A + B + C) | 1000 |

Manufacturing process: Similar to that described in Example 5.

### EXAMPLE 7

### Composition of Metoprolol Extended Release Tablets 200 mg

| | |
|---|---|
| | Qty per batch |
| | of 1000 tablets |
| Ingredient | (g) |

Drug loading

| | |
|---|---|
| Dicalcium phosphate anhydrous | 79.5 |
| Ethyl cellulose 10 cPS* | 3 |
| Metoprolol succinate | 190 |
| Hydroxypropyl methylcellulose, 5 cPS | 7.5 |
| Isopropyl alcohol | 50 |
| Methylene chloride | 50 |
| Water | 550 |
| Weight of drug-loaded pellets | 280 |

ER coating

| | |
|---|---|
| Ethyl cellulose, 10 cPS | 108 |
| Hydroxypropyl methylcellulose, 5 cPS | 32.5 |
| Triacetin NF | 26 |
| Isopropyl alcohol | 560 |
| Methylene chloride | 560 |
| Weight of ER coated pellets (A) | 447 |

Placebo granules

| | |
|---|---|
| Prosolv HD 90 (Silicified MCC) | 300 |
| Hydroxypropyl cellulose LF | 13 |
| Water | 540 |
| Croscarmellose sodium | 12 |
| Sodium stearyl fumarate | 4 |
| Colloidal silicon dioxide | 24 |
| Compression placebo weight (B) | 353 |
| Compressed tablet weight (mg) | 800 |

Film coating

| | |
|---|---|
| Hydroxypropyl methyl cellulose, 5 cPS | 16.6 |
| Polyethylene glycol 6000 | 24.8 |
| Talc | 2.1 |
| Titanium dioxide | 16.6 |
| Isopropyl alcohol | 500 |
| Methylene chloride | 500 |
| Film coating weight (C) | 60 |
| Theoretical weight of tablet (A + B + C) | 860 |

Manufacturing process: Similar to that described in Example 5.

### EXAMPLE 8

### Composition of Metoprolol Extended Release Tablets 200 mg

| | |
|---|---|
| | Qty per batch |
| | of 1000 tablets |
| Ingredient | (g) |

Drug loading

| | |
|---|---|
| Dicalcium phosphate anhydrous | 49.5 |
| Ethyl cellulose 10 cPS | 3 |
| Metoprolol succinate | 190 |
| Hydroxypropyl methylcellulose 5 cPS | 7.5 |
| Isopropyl alcohol | 60 |
| Methylene chloride | 60 |
| Water | 550 |
| Weight of drug-loaded pellets | 250 |

ER coating

| | |
|---|---|
| Ethyl cellulose 10 cPS | 149.3 |
| Hydroxypropyl methylcellulose 5 cPS | 44.8 |
| Acetyltributyl citrate | 35.8 |
| Isopropyl alcohol | 1200 |
| Methylene chloride | 1200 |
| Weight of ER coated pellets (A) | 480 |

Placebo granules

| | |
|---|---|
| Prosolv HD 90 (Silicified MCC) | 400.1 |
| Hydroxypropyl cellulose LF | 27.1 |
| Water | 500 |
| Croscarmellose sodium | 28.2 |
| Sodium stearyl fumarate | 4.7 |
| Compression placebo weight (B) | 460 |
| Compressed tablet weight (mg) | 940 |

Film coating

| | |
|---|---|
| Hydroxypropyl methylcellulose, 5 cPS | 16.6 |
| Polyethylene glycol 6000 | 24.8 |
| Talc | 2.1 |
| Titanium dioxide | 16.6 |
| Isopropyl alcohol | 500 |
| Methylene chloride | 500 |
| Film coating weight (C) | 60 |
| Theoretical weight of tablet (A + B + C) | 1000 |

Manufacturing process: Similar to that described in Example 5.

1. A pharmaceutical composition comprising an inert water-insoluble particle having a first coating comprising a drug substance, and optionally a second coating disposed over the first coating and containing a mixture of hydrophilic and hydrophobic polymers.

2. The pharmaceutical composition of claim 1, wherein an inert water-insoluble particle comprises a dibasic calcium phosphate.

3. The pharmaceutical composition of claim 1, wherein the first coating contains a hydrophilic polymer, a hydrophobic polymer, or a mixture thereof.

4. The pharmaceutical composition of claim 1, wherein a ratio of hydrophilic polymer to hydrophobic polymer in a second coating is 1:9 to 9:1.

5. The pharmaceutical composition of claim 1, wherein a ratio of hydrophilic polymer to hydrophobic polymer in a second coating is from 1:5 to 5:1.

6. The pharmaceutical composition of claim 1, wherein a ratio of hydrophilic polymer to hydrophobic polymer in a second coating is 1:3 to 3:1.

7. The pharmaceutical composition of claims 1, wherein a first coating contains a mixture of hydrophilic and hydrophobic polymers.

8. The pharmaceutical composition of claim 1, wherein a drug substance comprises metoprolol or a salt thereof.

9. The pharmaceutical composition of claim 1, wherein multiple coated particles are combined with at least one pharmaceutical excipient and compressed into a tablet.

10. The pharmaceutical composition of claim 1, wherein multiple coated particles are filled into a capsule.

11. A pharmaceutical composition comprising an inert water-insoluble particle having a first coating comprising metoprolol or a salt thereof, and optionally a hydrophilic polymer, a hydrophobic polymer, or a mixture thereof, and a second coating disposed over the first coating and containing a mixture of hydrophilic and hydrophobic polymers.

12. The pharmaceutical composition of claim 11, wherein an inert water-insoluble particle comprises a dibasic calcium phosphate.

13. The pharmaceutical composition of claim 11, wherein a first coating contains a mixture of hydrophilic and hydrophobic polymers.

14. The pharmaceutical composition of claim 11, wherein a ratio of hydrophilic polymer to hydrophobic polymer in a second coating is 1:9 to 9:1.

15. The pharmaceutical composition of claim 11, wherein a ratio of hydrophilic polymer to hydrophobic polymer in a second coating is from 1:5 to 5:1.

16. The pharmaceutical composition of claim 11, wherein a ratio of hydrophilic polymer to hydrophobic polymer in a second coating is 1:3 to 3:1.

17. The pharmaceutical composition of claim 11, wherein multiple coated particles are combined with at least one pharmaceutical excipient and compressed into a tablet.

18. The pharmaceutical composition of claim 11, wherein multiple coated particles are filled into a capsule.

19. A pharmaceutical composition comprising multiple particles comprising a dibasic calcium phosphate having a first coating comprising metoprolol or a salt thereof and a hydrophilic polymer, a hydrophobic polymer, or a mixture thereof, and a second coating disposed over the first coating and containing a mixture of hydrophilic and hydrophobic polymers.

20. The pharmaceutical composition of claim 19, wherein a first coating comprises a salt of metoprolol and a hydrophilic polymer, a hydrophobic polymer, or a mixture thereof.
